(19) United States
(12) Patent Application Publication
(10) Pub. No.: US 2012/0241537 A1
(43) Pub. Date: Sep. 27, 2012

(54) DISINFECTING SPRAY DEVICE FOR A CLEANING CART

(75) Inventors: Mark C. Schwei, Greenville, WI (US); Brad C. Schwei, Greenville, WI (US)

(73) Assignee: PURETECH SYSTEMS, LLC, Neenah, WI (US)

(21) Appl. No.: 13/427,526

(22) Filed: Mar. 22, 2012

Related U.S. Application Data
(60) Provisional application No. 61/466,093, filed on Mar. 22, 2011.

(51) Int. Cl. B05B 9/00; A61L 2/18

(52) U.S. Cl. 239/147; 422/300

(57) ABSTRACT

A disinfecting spray module that can either be retrofit to an existing housekeeping cart or integrated into the housekeeping cart. The disinfecting spray module includes a storage container for receiving a disinfectant or cleaner. An electric pump withdraws the disinfectant and supplies the pump through a spray hose. The spray hose includes a handle having a nozzle to direct the disinfectant onto the surface to be cleaned. The disinfecting spray module can include an internal power supply or be connected to a source of electricity within the room being disinfected.

### CROSS-REFERENCE TO RELATED APPLICATION

The present application is based on and claims priority to U.S. Provisional Patent Application Ser. No. 61/466,093 filed Mar. 22, 2011.

### BACKGROUND

The present disclosure generally relates to a disinfecting spray module that can be utilized to sanitize equipment and disinfect and/or clean rooms. More specifically, the present disclosure relates to a disinfecting spray module that can be retrofit onto existing housekeeping carts or other types of mobile carts to provide a supply of disinfectant that can be evenly sprayed onto a surface to be cleaned.

Presently, housekeeping staff typically carries one or more spray bottles of disinfectant on a housekeeping cart. The housekeeping staff utilizes the spray bottles to spray the disinfectant onto surfaces that need to be cleaned. Although a spray bottle is generally adequate to apply disinfectant to the surface to be cleaned, the size of the spray bottle is limited which requires the housekeeping staff to refill the bottle frequently or carry multiple bottles on the housekeeping cart. Additionally, spray bottles do not generally apply the disinfectant/cleaner in a smooth, continuous pattern.

In addition to the limited supply of disinfectant that can be carried in a portable spray bottle, the distribution pattern of the disinfectant onto the surface to be cleaned is inconsistent and varied during the extended use of the spray bottle. The coverage and overall distribution of the disinfectant is affected by the type of nozzle used on the spray bottle and the action of the cleaning personnel utilizing the spray bottle.

Therefore, a need exists for an improved system that provides a larger supply of disinfectant and allows the housekeeping staff to more effectively apply the disinfectant or cleaner onto the surfaces to be cleaned.

### SUMMARY

The present disclosure relates to a small, portable spray module that can be retrofitted to existing housekeeping carts or other types of mobile carts, such as luggage carts, or pre-assembled on new housekeeping carts. The small, portable spray module is used to disinfect rooms, equipment, solid surfaces and a variety of other surfaces such as fabric and wheelchairs.

The disinfecting spray module is configured to apply any disinfectant or cleaner evenly and accurately over a desired surface. The disinfecting spray module creates a fine spray that allows the solution to penetrate hard-to-reach areas and dries in minutes.

The disinfecting spray module includes an electric pump that is virtually silent and is either connected to a power supply in the room being cleaned or includes a rechargeable battery pack. The disinfecting spray module can be mounted to a housekeeping cart or a simple luggage cart such that the spray module is portable and can be easily moved throughout a facility. The disinfecting spray module does not take up any additional inner-cart space and thus maintains optimal storage on the cart.

The disinfecting spray module includes an easy-to-use dispensing system and applies any disinfectant or cleaner within seconds. The disinfecting spray module includes a storage container that can be accessed by simply unscrewing a fill cap and filling the container with any desired solution.

The disinfecting spray module of the present disclosure has been found to provide labor savings of up to 50%, reduces FTEs due to the mobility and ease of use. The disinfecting spray module is effective at applying a disinfectant to hard-to-reach areas that may be overlooked with traditional methods of disinfectant.

The disinfecting spray module of the present disclosure has been designed to include a spray nozzle that creates a fine mist of the disinfectant while insuring that the disinfectant is not released in a mist. The selection of the nozzle design creates the desired droplet size and the desired pattern of coverage while controlling the size of the droplets to prevent the creation of a mist, which can be inhaled.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a perspective view of a cleaning cart with a disinfecting spray module;

FIG. 2 is a front perspective view of an alternate type of cleaning cart also incorporating the disinfecting spray module of the present disclosure;

FIG. 3 is a front perspective view of another alternate type of cart that can be used to support and transport the disinfecting spray module of the present disclosure;

FIG. 4 is a perspective view of the disinfecting spray module;

FIG. 5 is a perspective view similar to FIG. 4 with the outer housing of the module removed; and

FIG. 6 is a front view of the operating components of the disinfecting spray module shown in FIG. 5.

### DETAILED DESCRIPTION OF THE INVENTION

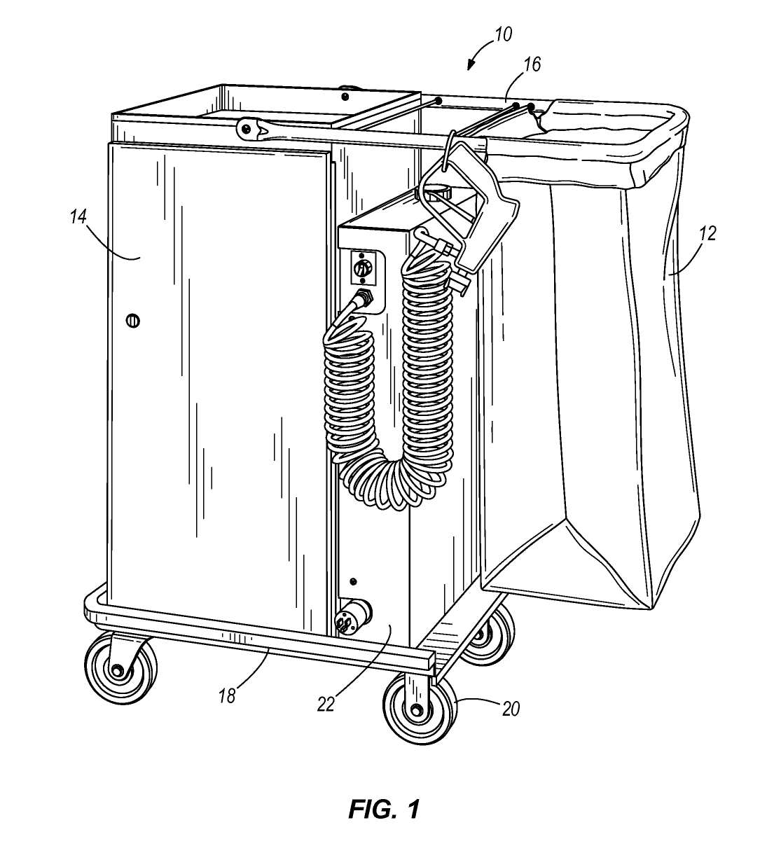

FIG. 1 illustrates a housekeeping cart 10 that is typically used by cleaning personnel to disinfect or clean rooms, equipment and solid surfaces. The housekeeping cart 10 includes a waste receptacle 12 supported on a storage cabinet 14 by a pair of support arms 16. The storage cabinet 14 is mounted to a platform 18 having a series of caster wheels 20 such that the entire housekeeping cart 10 is mobile and can be moved from room to room by housekeeping personnel. In accordance with the present disclosure, the housekeeping cart 10 is configured to include a removably mounted disinfecting spray module 22 constructed in accordance with the present disclosure.

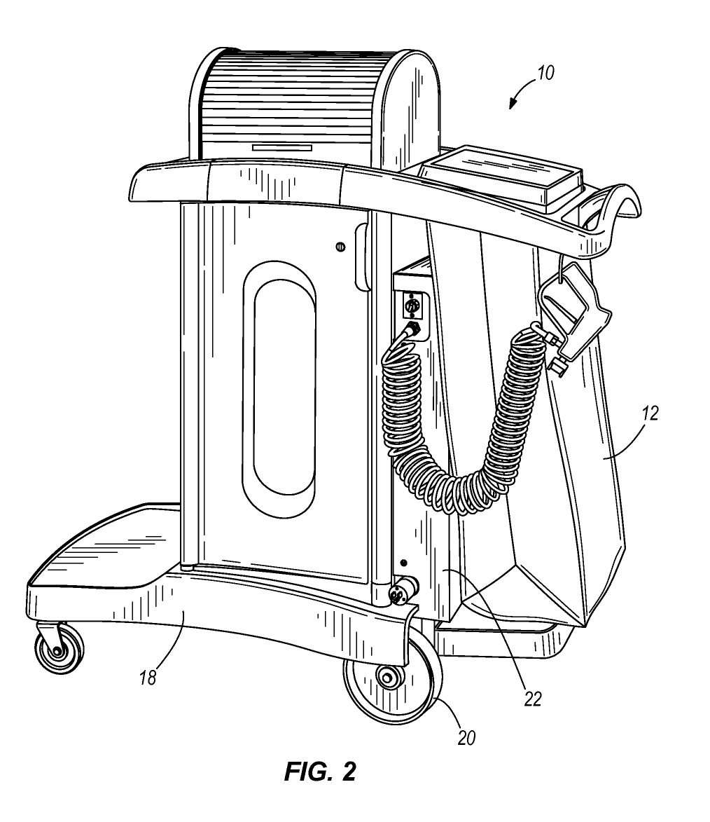

FIG. 2 illustrates another common type of housekeeping cart 10 that includes a similar waste receptacle 12 and platform 18 having the series of caster wheels 20. As illustrated in FIG. 2, the disinfecting spray module 22 is supported by the platform 18 in a similar manner to the embodiment shown in FIG. 1. It should be understood by a comparison between FIGS. 1 and 2 that various different types of housekeeping carts 10 can be utilized to support the disinfecting spray module 22 of the present disclosure. In the embodiments of FIGS. 1 and 2, the disinfecting spray module 22 is retrofit onto an existing cart. However, it is contemplated that the housekeeping cart 10 could be specifically designed to include the disinfecting spray module 22.

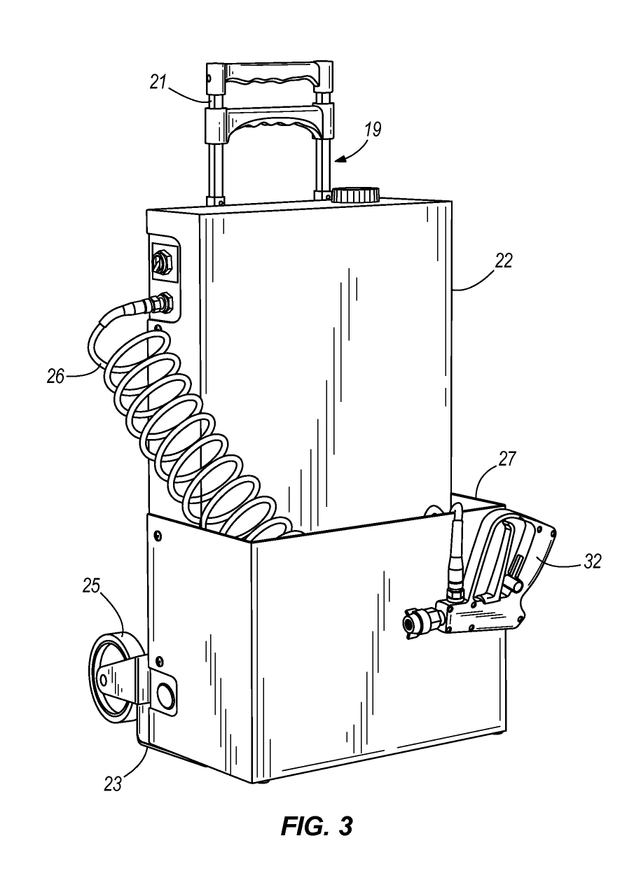

FIG. 3 illustrates yet another configuration and use for the disinfecting spray module 22 constructed in accordance with the present disclosure. In the embodiment shown in FIG. 3, the disinfecting spray module 22 is shown mounted to a conventional luggage cart 19 that includes an extendible handle 21 and a lower support platform 23. A pair of wheels 25 allow the luggage cart 19 to support and move the spray module 22 to a desired location. In the embodiment shown in FIG. 3, a storage bin 27 is mounted to extend from the front face of the disinfecting spray module 22. The storage bin 27 provides a convenient storage location for various different tools needed by cleaning personnel and also provides a storage location for a spray handle 32 positioned at the end of the spray hose 26. In the embodiment shown, the front storage container 27 is formed from stainless steel, although other materials, such as plastic, are contemplated as being within the scope of the present disclosure.

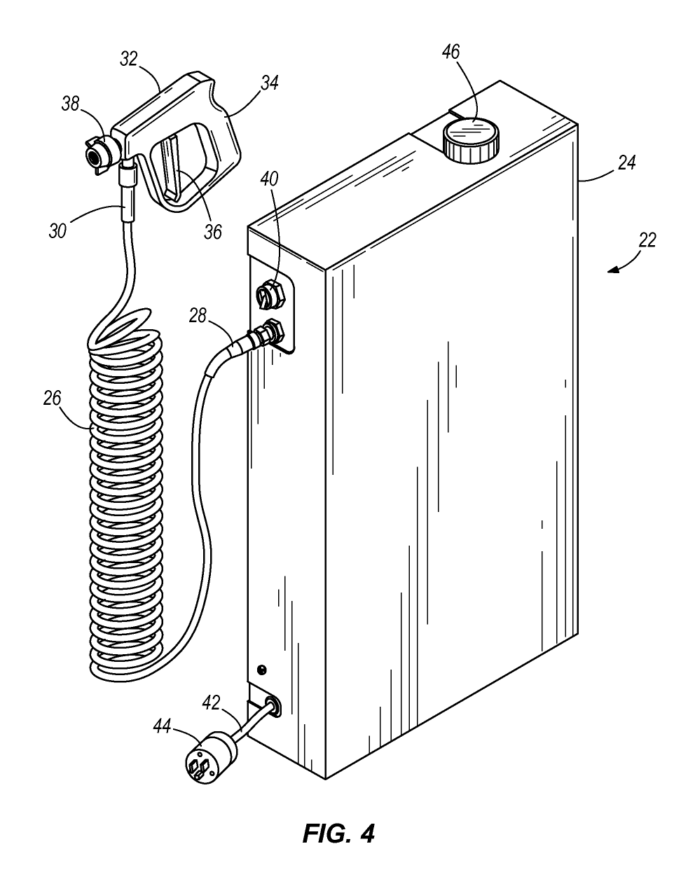

FIG. 4 illustrates the disinfecting spray module 22 constructed in accordance with the present disclosure. The disinfecting spray module 22 includes an outer housing 24 preferably formed from stainless steel. Stainless steel is a desirable material for the outer housing 24 since stainless steel can be easily sanitized and provides the required durability.

The disinfecting spray module 22 includes a coiled spray hose 26 having a first end 28 connected to the spray module 22. A second end 30 of the spray hose 26 is connected to a spray handle 32. The spray handle 32 includes a handle 34, trigger 36 and spray nozzle 38.

The housing 24 further includes an on/off switch 40 that allows an operator to control activation of the disinfecting spray module 22. A power cord 42 having a plug 44 extends from the housing 24. A fill cap 46 is accessible through the top of the outer housing 24. The fill cap 46 provides an access point for filling the disinfecting spray module 22 with any hospital-grade disinfectant.

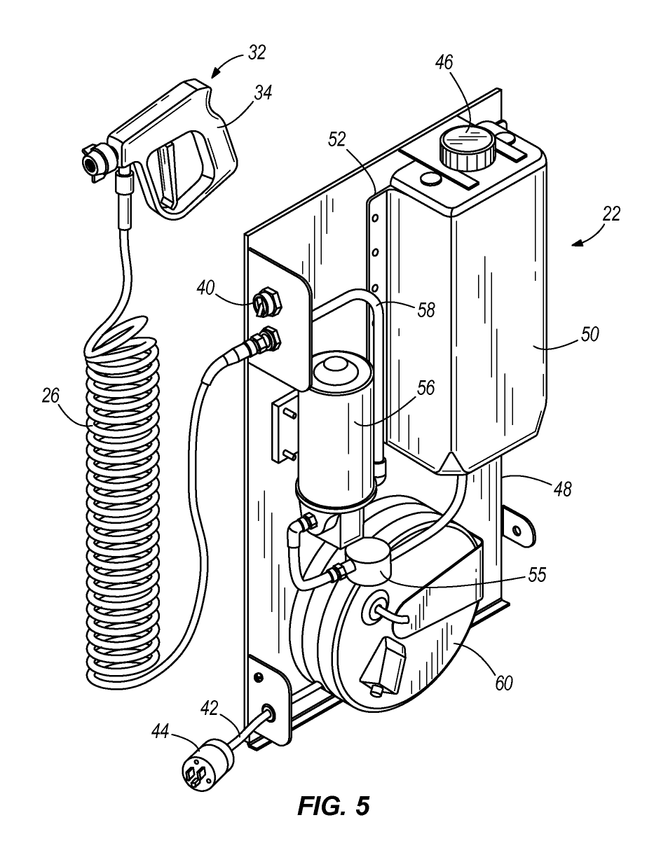

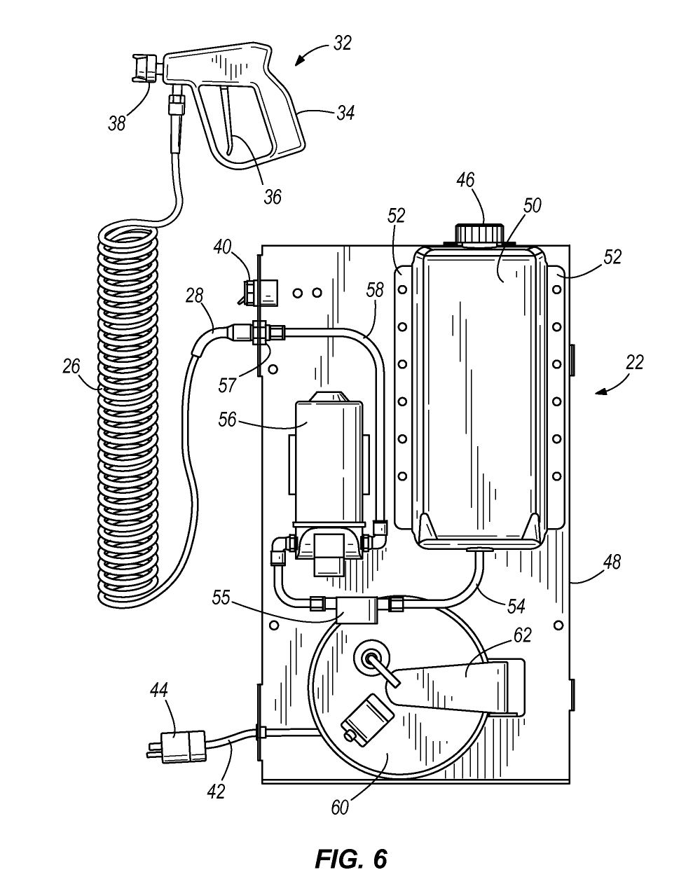

FIGS. 5 and 6 illustrate the operating components of the disinfecting spray module 22 with the outer housing removed. The disinfecting spray module 22 includes a mounting board 48 formed from stainless steel. The mounting board 48 provides a convenient point of attachment for the remaining operating components of the disinfecting spray module 22.

A liquid container 50 is attached to the mounting board 48 by a pair of attachment flanges 52. The attachment flanges 52 support the liquid container and are preferably formed from a metal material. The liquid container 50 may be formed from various different materials, but is shown in the embodiments of FIGS. 5 and 6 as being formed from a molded plastic. The liquid container 50 receives the fill cap 46 which allows the liquid disinfectant or cleaner to be poured into the open interior of the liquid container.

As best illustrated in FIG. 6, an outlet tube 54 extends from a bottom end of the liquid container 50 to an electric pump 56 through a strainer 55. The electric pump 56 is operable to draw the liquid disinfectant from the liquid container 50 and supply the liquid disinfectant to the spray hose 26. A discharge tube 58 extends from the pump 56 and is connected to the first end 28 of the spray hose 26 through a fitting 57.

In the embodiment shown in FIGS. 5 and 6, the pump 56 includes an electric drive motor that receives a supply of power from an electronic outlet within the facility being cleaned. Plug 44 contained on the power cord 42 provides the point of connection between the disinfecting spray module 22 and the main power supply for the facility. Power cord 42 is shown in the embodiments of FIGS. 5 and 6 as stored on a reel 60. The reel 60 is spring-biased and retracts the power cord 42 and can hold up to twenty-five feet of power cord. The reel 60 is mounted to the mounting board 48 by a support bracket 62 such that the reel 60 can rotate to dispense and accumulate the power cord 42.

Although the embodiment shown in FIGS. 5 and 6 includes the retractable power cord that connects to a 110 volt GFI grounded electrical outlet, the disinfecting spray module could be configured to include a rechargeable battery pack that could run the pump 56 for up to eight hours. In such an embodiment, the battery pack would be positioned within the open interior defined by the outer housing and have a recharging terminal or terminals that would extend through the housing. Various different types of rechargeable batteries are contemplated as being within the scope of the present disclosure.

In the embodiment shown in the drawing Figures, the liquid container 50 is sized to hold approximately 1.5 gallons liters of disinfectant, although other sizes for the liquid container are contemplated as being within the scope of the present disclosure.

During operation, when an operator squeezes the trigger 36, the built-up pressure of liquid within the spray hose 26 causes the disinfectant to be initially sprayed out of the spray nozzle 38. As the pressure within the hose 26 decreases, the pump 56 senses the pressure drop and begins to operate. Operation of the electric motor in the pump 56 causes an additional supply of fluid to be drawn from the container 50 and supplied to the spray hose 26 and ultimately through the nozzle 38.

As illustrated in FIG. 6, the spray handle 32 includes a nozzle 38 that controls both the spray pattern and the droplet size of the spray of disinfecting solution from the spray module 22. The specific design and configuration of the nozzle 38 was selected to insure that the liquid being dispensed from the disinfecting spray module 22 is dispensed in a fine spray and to avoid the creation of a mist. Since the disinfecting spray module 22 will be used in many different environments, including within a hospital, it is important to prevent the creation of a mist, since a mist can be inhaled by patients and cleaning personnel. Therefore, the nozzle 38 was specifically selected to create a desired size of droplets and spray patterns.

In the embodiment shown in FIG. 6, the nozzle 38 includes a spray tip that creates a flat fan spray pattern with uniform distribution. At 40 psi, the nozzle creates a flat spray having a 65° spray pattern. Although a specific nozzle is described in the present disclosure, it should be understood that various different types of nozzles could be utilized while operating within the scope of the present disclosure. However, it is important that the nozzle size be selected to create a fine spray of liquid and not to create a mist.

In accordance with the present disclosure, the nozzle 38 is selected such that at 40 psi, the nozzle creates a spray having droplet sizes preferably between 110 μm and 2500 μm. In the range of 100-2500 μm, the droplet size discharged from the nozzle 38 creates a fine spray and does not create a mist. As indicated above, it is important that the nozzle 38 create a fine spray rather than a mist to prevent uncontrolled inhalation and distribution of the mist in the area around the nozzle 38.

As illustrated in FIG. 4, the disinfecting spray module 22 is a self-contained unit that can be either mounted to the various types of cleaning carts shown in FIGS. 1 and 2 or a luggage cart as shown in FIG. 3. Alternatively, the disinfecting spray module 22 can also be mounted at a stationary location, such as on a wall of a room where various products or equipment are disinfected on a regular basis. The self-contained disinfecting spray module 22 includes the self-contained cord such that the spray module 22 can be connected to a supply of power. Alternatively, a battery pack can be connected to the disinfecting spray module 22 to allow the spray module 22 to be driven by a rechargeable battery pack.

The extendible, coiled hose 26 shown in the drawing Figures allows for a great range of movement of the spray handle 32 away from the module 22. The nozzle 38 is selected to control the size of spray droplets from the disinfecting spray module 22 while providing a uniform, wide degree of spray coverage.

In the preferred embodiment shown in the drawing Figures, the electric pump 56 was selected to both optimize the battery life and minimize the amount of noise created by the disinfecting spray module 22. Since the spray module 22 is often used in a quiet, hospital environment, it is important that the electric motor that drives the pump 56 operate quietly to reduce the amount of noise created by the spray module 22. Although various types of electric motors and pumps can be utilized while operating within the scope of the present disclosure, the specific electric motor selected minimizes the amount of noise created while providing the required pressure for distributing the disinfecting solution.

We claim:

1. A disinfecting spray module for use in disinfecting a surface or article, comprising:
an outer housing;
a storage container positioned within the outer housing and having an internal volume to receive a supply of disinfecting solution;
an electric pump contained within the outer housing and in fluid communication with the storage container, wherein the electric pump withdraws the supply of disinfecting solution from the storage container upon activation;
a supply hose extending from the outer housing and having a first end in fluid communication with the electric pump to receive the supply of disinfecting solution upon activation of the electric pump;
a spray handle connected to a second end of the supply hose; and
a spray nozzle mounted to the spray handle, wherein the spray nozzle is sized to create a spray of disinfecting solution upon activation of the electric pump.

2. The spray module of claim 1 wherein the electric pump receives power from a power cord contained within and retractable from the outer housing.

3. The spray module of claim 2 wherein the power cord is wound around a reel mounted for rotation within the outer housing.

4. The spray module of claim 3 wherein the storage container, the electric pump and the reel are attached to a mounting board sized to be received within the outer housing.

5. The spray module of claim 1 wherein the electric pump receives power from a rechargeable battery pack.

6. The spray module of claim 5 wherein the rechargeable battery pack is contained within the outer housing.

7. The spray module of claim 1 further comprising a storage bin mounted to the outer housing and sized to receive the supply hose and the spray handle.

8. The spray module of claim 1 wherein the spray module is self-contained.

9. The spray module of claim 1 wherein the outer housing is formed from stainless steel.

10. The spray module of claim 1 wherein the spray of disinfecting solution includes spray droplets between 110-2500 μm.

11. A cleaning cart for use in disinfecting a surface or an article comprising:
a cart having a support platform and a plurality of wheels; and
a spray module supported on the support platform, the spray module comprising:
an outer housing;
a storage container positioned within the outer housing and having an internal volume to receive a supply of disinfecting solution;
an electric pump contained within the outer housing and in fluid communication with the storage container, wherein the electric pump withdraws the supply of disinfecting solution from the storage container upon activation;
a supply hose extending from the outer housing and having a first end in fluid communication with the electric pump to receive the supply of disinfecting solution upon activation of the electric pump;
a spray handle connected to a second end of the supply hose; and
a spray nozzle mounted to the spray handle, wherein the spray nozzle is sized to create a spray of disinfecting solution upon activation of the electric pump.

12. The cleaning cart of claim 11 wherein the cart further includes a waste receptacle.

13. The cleaning cart of claim 11 wherein the electric pump receives power from a power cord contained within and retractable from the outer housing.

14. The cleaning cart of claim 13 wherein the power cord is wound around a reel mounted for rotation within the outer housing.

15. The cleaning cart of claim 14 wherein the storage container, the electric pump and the reel are attached to a mounting board sized to be received within the outer housing.

16. The cleaning cart of claim 11 wherein the electric pump receives power from a rechargeable battery pack contained within the outer housing.

17. The cleaning cart of claim 11 further comprising a storage bin mounted to the outer housing and sized to receive the supply hose and the spray handle.

18. The cleaning cart of claim 11 wherein the spray module is self-contained.

19. The cleaning cart of claim 11 wherein the outer housing is formed from stainless steel.

20. The cleaning cart of claim 11 wherein the spray of disinfecting solution includes spray droplets between 110-2500 μm.
